(19) United States
(12) Patent Application Publication
(10) Pub. No.: US 2024/0050457 A1
(43) Pub. Date: Feb. 15, 2024

(54) METHODS OF MONITORING DIGOXIN WITH CONCOMITANT USE OF VIBEGRON TO TREAT OVERACTIVE BLADDER

(71) Applicant: UROVANT SCIENCES GMBH, Zug (CH)

(72) Inventor: Paul N. MUDD, Jr., Irvine, CA (US)

(21) Appl. No.: 18/258,953

(22) PCT Filed: Dec. 22, 2021
(86) PCT No.: PCT/IB2021/062208

§ 371 Date: Jun. 22, 2023

Related U.S. Application Data
(60) Provisional application No. 63/129,474, filed on Dec. 22, 2020.

(51) Int. Cl. A61K 31/7048; A61K 31/519

(52) U.S. Cl. CPC A61K 31/7048; A61K 31/519

(57) ABSTRACT

The present disclosure is directed to a method of treating overactive bladder comprising orally administering to a patient in need thereof a therapeutically effective amount of vibegron, wherein the patient is concomitantly receiving digoxin, wherein the patient's digoxin serum level is maintained at an amount that results in a desired digoxin clinical effect. This can be achieved by monitoring the patient's digoxin serum level before, during, and/or after vibegron treatment, and either maintaining or titrating the digoxin dose based on that level to achieve the desired clinical effect.

### CROSS-REFERENCE TO RELATED APPLICATIONS

This application claims the priority benefit of U.S. Provisional Application No. 63/129,474, filed Dec. 22, 2020, which is incorporated by reference herein in its entirety.

### BACKGROUND

Overactive bladder (OAB) is a chronic and sometimes debilitating condition of the lower urinary tract. The function of the lower urinary tract is to store and periodically release urine. This requires the orchestration of storage and micturition reflexes which involve a variety of afferent and efferent neural pathways, leading to modulation of central and peripheral neuroeffector mechanisms, and resultant coordinated regulation of sympathetic and parasympathetic components of the autonomic nervous system as well as somatic motor pathways. These proximally regulate the contractile state of bladder (detrusor) and urethral smooth muscle, and urethral sphincter striated muscle.

Vibegron, (6S)—N-[4-[[(2S,5R)-5-[(R)-hydroxy(phenyl)methyl]pyrrolidin-2-yl]methyl]phenyl]-4-oxo-7,8-dihydro-6H-pyrrolo[1,2-a]pyrimidine-6-carboxamide, is a potent and highly selective beta-3 adrenergic receptor agonist demonstrating >9,000 fold selectivity for activation of β₃-AR over β₂-AR and β₁-AR in cell based in vitro assays. See Edmondson et al., J. Med. Chem. 59:609-623 (2016).

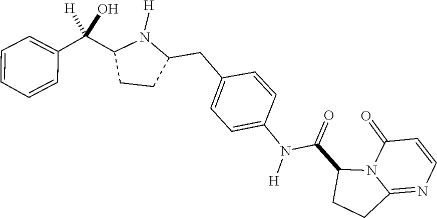 [SMILES: O=C(Nc1ccc(C[C@@H]2CC[C@H]([C@H](O)c3ccccc3)N2)cc1)[C@@H]1CCc2nccc(=O)n21]

Vibegron is disclosed as a β3-AR agonist in U.S. Pat. Nos. 8,399,480, 8,653,260, and 8,247,415. Synthetic methods for preparing vibegron are disclosed in United States Publication Nos. US 2017/0145014, US 2015/0087832, US 2016/0176884 and US 2014/0242645. All of the cited publications are herein incorporated by reference in their entireties.

As vibegron is not an inhibitor of any major enzymes produced from the cytochrome P450 genes, its combined use with drugs metabolized by these enzymes has not led to clinically significant drug-drug interactions (See, for example, Rechberger, T. et al. (2020): Evaluating vibegron for the treatment of overactive bladder, Expert Opinion on Pharmacotherapy, DOI: 10. 1080/14656566.2020. 1809652). WO2018/224989 discloses the evaluation of multiple doses of vibegron in combination with the p-gp substrate digoxin and suggests that vibegron does not influence digoxin pharmacokinetics to a clinically significant degree. WO2018/224989 neither discloses nor suggests that monitoring of serum digoxin concentrations in patients and/or titration of the digoxin dose might be necessary or desirable.

However, despite the belief that vibegron does not cause these types of interactions, there is concern that the use of vibegron in patients taking drugs that have a sensitive pharmacokinetic profile may alter the therapeutic effect of those drugs. Therefore, there is a need to ensure that co-administration of vibegron with certain drugs will not impact the clinical effect of those drugs. This need can be achieved by ensuring that the blood serum level of a drug concomitantly administered with vibegron is one that results in a desired clinical effect. This can be achieved by monitoring the serum levels of certain co-administered drugs before, during, and/or after co-administration with vibegron, and based on those serum levels, maintaining or titrating the dosage of the co-administered drug to obtain the desired clinical effect.

### BRIEF DESCRIPTION OF THE DRAWINGS

FIG. 1 depicts patient serum digoxin concentration versus collection time points for Treatment A.

FIG. 2 depicts patient serum digoxin concentration versus collection time points for Treatment B.

### SUMMARY

The present disclosure provides a method of treating overactive bladder, the method comprising orally administering to a patient in need thereof a therapeutically effective amount of vibegron, wherein the patient is concomitantly receiving digoxin, wherein the patient's digoxin serum level is maintained at a level that can achieve the desired clinical effect. This can be achieved by monitoring the patient's serum digoxin level before, during, and/or after vibegron treatment, and based on that serum level, maintaining or titrating the patient's digoxin dose to achieve the desired clinical effect.

In some aspects, the disclosure provides a method of treating overactive bladder, the method comprising orally administering to a patient in need thereof a therapeutically effective amount of vibegron, wherein the patient is concomitantly receiving digoxin, and wherein the method further comprises:
a. monitoring serum digoxin concentrations in the patient;
b. in response to said monitoring of step (a), either maintaining or titrating the digoxin dose in the patient to obtain a desired clinical effect;
c. discontinuing administration of vibegron;
d. monitoring serum digoxin concentrations in the patient after said discontinuation; and
e. in response to said monitoring of step (d), either maintaining or titrating the digoxin dose in the patient to obtain a desired clinical effect.

In some aspects, the disclosure provides a method of treating overactive bladder, the method comprising orally administering to a patient in need thereof a therapeutically effective amount of vibegron, wherein the patient is concomitantly receiving digoxin, and wherein the patient's digoxin dose is titrated based on the patient's serum digoxin concentration.

In some aspects, the disclosure provides a method of treating overactive bladder, the method comprising orally administering to a patient in need thereof a therapeutically effective amount of vibegron, wherein the patient is concomitantly treated with digoxin, and wherein the patient's digoxin serum level is one that results in a desired digoxin clinical effect.

In some aspects, the therapeutically effective amount of vibegron is between about 50 mg/day and about 150 mg/day.

In some aspects, the therapeutically effective amount of vibegron is about 50 mg/day, about 75 mg/day, about 100 mg/day, or about 150 mg/day. In some aspects, the therapeutically effective amount of vibegron is 75 mg/day.

In some aspects, the patient has a symptom selected from the group of urgency urinary incontinence, urinary urgency, urinary frequency, and combinations thereof. In some aspects, the patient has the symptoms of urgency urinary incontinence, urinary urgency, and urinary frequency.

In some aspects, the patient is a human. In some aspects, the human is a female. In some aspects, the human is a male. In some aspects, the human is over the age of 65 years.

In some aspects, the vibegron is administered once per day. In some aspects, vibegron is administered with a meal. In some aspects, vibegron is administered without a meal.

In some aspects, vibegron is administered as a free base. In some aspects, vibegron is administered as a pharmaceutically acceptable salt thereof.

In some aspects, vibegron has onset of action of about 4 weeks, about 3 weeks, or about 2 weeks.

In some aspects, the present disclosure provides a method of treating overactive bladder, the method comprising orally administering to a patient in need thereof 75 mg of vibegron per day, wherein the patient is concomitantly receiving digoxin, and wherein the method further comprises:
a. monitoring serum digoxin concentrations in the patient; and
b. in response to said monitoring, either maintaining or titrating the digoxin dose in the patient to obtain a desired clinical effect.

In some aspects, the present disclosure provides a method of treating overactive bladder, the method comprising orally administering to a patient in need thereof 75 mg of vibegron per day, wherein the patient is concomitantly receiving digoxin, and wherein the method further comprises:
a. monitoring serum digoxin concentrations in the patient;
b. in response to said monitoring of step (a), either maintaining or titrating the digoxin dose in the patient to obtain a desired clinical effect;
c. discontinuing administration of vibegron;
d. monitoring serum digoxin concentrations in the patient after said discontinuation; and
e. in response to said monitoring of step (d), either maintaining or titrating the digoxin dose in the patient to obtain a desired clinical effect.

In some aspects, the present disclosure provides a method of treating overactive bladder, the method comprising orally administering to a patient in need thereof 75 mg of vibegron per day, wherein the patient is concomitantly receiving digoxin, and wherein the patient's digoxin dose is titrated based on the patient's serum digoxin concentration.

In some aspects, the present disclosure provides a method of treating overactive bladder, the method comprising orally administering to a patient in need thereof 75 mg of vibegron per day, wherein the patient is concomitantly treated with digoxin, and wherein the patient's digoxin serum level is one that results in a desired digoxin clinical effect.

In some aspects, the present disclosure provides a method of treating overactive bladder in a patient, the method comprising:
a. measuring serum digoxin concentrations in the patient;
b. orally administering to the patient 75 mg of vibegron per day, wherein the patient is concomitantly receiving digoxin;
c. monitoring serum digoxin concentrations in the patient; and
d. in response to said monitoring of step (c), either maintaining or titrating the digoxin dose in the patient to obtain a desired clinical effect.

In some aspects, the present disclosure provides a method of treating overactive bladder in a patient, the method comprising:
a. measuring serum digoxin concentrations in the patient;
b. orally administering to the patient 75 mg of vibegron per day, wherein the patient is concomitantly receiving digoxin;
c. monitoring serum digoxin concentrations in the patient;
d. in response to said monitoring of step (c), either maintaining or titrating the digoxin dose in the patient to obtain a desired clinical effect;
e. discontinuing administration of vibegron;
f. monitoring serum digoxin concentrations in the patient after said discontinuation; and
g. in response to said monitoring of step (f), either maintaining or titrating the digoxin dose in the patient to obtain a desired clinical effect.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

The term “about” as used in connection with a numerical value throughout the specification and the claims denotes an interval of accuracy, familiar and acceptable to a person skilled in the art. In certain aspects, such interval of accuracy is +10%. In other aspects, such interval of accuracy is +5%.

The term “overactive bladder” generally refers to urinary urgency, usually accompanied by frequency and nocturia, with or without urgency urinary incontinence, in the absence of urinary tract infection or other obvious pathology. The term “overactive bladder” is defined by the International Continence Society (ICS) as follows: Overactive bladder (OAB) is a symptom complex consisting of urgency with or without urge incontinence, usually with frequency and nocturia, in the absence of local pathologic or hormonal factors (Abrams P et al., Urology 2003, 61(1): 37-49; Abrams P et al., Urology 2003, 62(Supplement 5B): 28-37 and 40-42). Synonyms of overactive bladder (OAB) include “urge syndrome” and “urge frequency syndrome.”

The term “urge incontinence” refers to a complaint of involuntary loss of urine.

The term “urgency urinary incontinence” (UUI) refers to a complaint of involuntary loss of urine associated with urgency and can be used interchangeably with “urge urinary incontinence” or “urge incontinence.” UUI is distinguished from stress urinary incontinence, which is the involuntary loss of urine on effort or physical exertion (e.g., sporting activities), or on sneezing or coughing.

The term “urinary urgency” as used herein means a sudden compelling desire to urinate which is difficult to defer.

The term “urinary frequency” as used herein refers to a need for frequent emptying of the bladder.

The term “free base” as used herein refers to a basic chemical compound itself, not in the form of a salt. For example, vibegron free base refers to (6S)—N-[4-[[(2S,5R)-5-[(R)-hydroxy(phenyl)methyl]pyrrolidin-2-yl]methyl]phenyl]-4-oxo-7,8-dihydro-6H-pyrrolo[1,2-a]pyrimidine-6-carboxamide.

The term “pharmaceutically acceptable salt” means those salts of compounds that are safe and effective for use in patients and that possess the desired biological activity.

Pharmaceutically acceptable salts of a basic compound can be salts of organic or inorganic acids. In some aspects, the organic and inorganic acids include but are not limited to hydrochloric acid, hydrobromic acid, sulfuric acid, phosphoric acid, citric acid, maleic acid, mandelic acid, succinic acid and methanesulfonic acid. See generally, Journal of Pharmaceutical Science, 66, 2 (1977), which is incorporated herein by reference in its entirety.

The term “Cₘₐₓ” as used herein refers to the maximum plasma concentration of a drug after it is administered.

The term “Tₘₐₓ” as used herein refers to the time after administration of a drug when the maximum plasma concentration is reached.

The term “AUC” as used herein refers to the area under the curve of a plot of plasma concentration versus time following administration of a drug.

The term “desired clinical effect” means the response after administration of a therapeutic agent, the results of which are useful or favorable to a clinical effect. In certain aspects, the term “desired clinical effect” refers to the response after administration of digoxin. In some aspects, the desired clinical effect obtained in a patient treated with digoxin includes, but is not limited to, reducing or eliminating irregular heartbeat (including atrial fibrillation), and/or reducing or ameliorating the symptoms of heart failure.

As used herein, the terms “measure” or “measuring” refer to the process of determining blood serum levels of a therapeutic agent. In certain aspects the terms refer to the process of determining blood serum levels of digoxin.

As used herein, the terms “monitor” or “monitoring” refer to the process of determining blood serum levels of a therapeutic agent at certain time intervals. In some aspects the process is conducted daily. In some aspects, the process is conducted weekly. In some aspects, the process is conducted bi-weekly. In some aspects, the process is conducted monthly. In certain aspects the terms refer to the process of determining blood serum levels of digoxin.

As used herein, the terms “treated,” “treating,” or “treatment” or “therapy” refer to partially or completely alleviating, ameliorating, improving, relieving, delaying onset of, inhibiting progression of, reducing severity of, reducing incidence of one or more symptoms or features of disease, or any combination thereof.

As used herein, the terms “patient,” and “patients” are synonymous with and can be used interchangeably with “subject” and “subjects.”

As used herein, the term “concomitantly” is synonymous with and can be used interchangeably with “simultaneously.”

As used herein, the terms “serum digoxin concentration,” “serum digoxin level,” and “digoxin serum level,” are synonymous and can be used interchangeably.

In general, the term “treatment” refers to countering the effects caused as a result of the disease or pathological condition of interest in a patient including (i) inhibiting the progress of the disease or pathological condition, in other words, slowing or stopping the development or progression thereof, or one or more symptoms of such disorder or condition; (ii) relieving the disease or pathological condition, in other words, causing said disease or pathological condition, or the symptoms thereof, to regress; (iii) stabilizing the disease or pathological condition or one or more symptoms of such disorder or condition, (iv) reversing the disease or pathological condition or one or more symptoms of such disorder or condition to a normal state, (v) preventing the disease or pathological condition or one or more symptoms of such disorder or condition, and (vi) any combination thereof.

### Methods of Treatment

In certain aspects, the present disclosure provides a method of treating overactive bladder, the method comprising orally administering to a patient in need thereof a therapeutically effective amount of vibegron, wherein the patient is concomitantly receiving digoxin, and wherein the method further comprises: monitoring serum digoxin concentrations in the patient; and in response to said monitoring, either maintaining or titrating the digoxin dose in the patient to obtain a desired clinical effect.

In some aspects, the present disclosure provides a method of treating overactive bladder, the method comprising orally administering to a patient in need thereof a therapeutically effective amount of vibegron, wherein the patient is also receiving digoxin, and wherein the method further comprises monitoring serum digoxin concentrations in the patient; and titrating the digoxin dose in the patient to obtain a desired clinical effect.

In some aspects, the present disclosure provides a method of treating overactive bladder, the method comprising orally administering to a patient in need thereof a therapeutically effective amount of vibegron, wherein the patient is concomitantly treated with digoxin, and wherein the patient's digoxin serum level is one that results in a desired digoxin clinical effect.

In some aspects, the digoxin is administered to the patient prior to the vibegron. In some aspects, the digoxin is administered to the patient after the vibegron. In some aspects, the digoxin is administered to the patient simultaneously with the vibegron.

In some aspects, the patient's digoxin serum level is monitored before vibegron is co-administered with the digoxin. In some aspects, the patient's digoxin serum level is monitored during the period in which vibegron is co-administered with the digoxin. In some aspects, the patient's digoxin serum level is monitored after the patient stops taking vibegron. In some aspects, the patient's digoxin serum level is monitored before, during, and/or after the vibegron is co-administered with the digoxin.

In some aspects, the patient's digoxin serum level is measured before initiating co-administration of vibegron with digoxin and is monitored during the period in which vibegron is co-administered with the digoxin to titrate the digoxin dose to the desired clinical effect.

In some aspects the patient's serum digoxin concentrations are monitored before initiating and during therapy with vibegron and used for titration of the digoxin dose to obtain the desired clinical effect. In some aspects digoxin concentration monitoring continues upon discontinuation of vibegron and the digoxin dose adjusted as needed.

In some aspects, the amount of vibegron administered per day is from about 50 mg to about 150 mg. In some aspects, the amount of vibegron administered per day is from about 70 mg to about 80 mg. In some aspects, the amount of vibegron administered per day is about 50 mg, about 75 mg, about 100 mg, about 125 mg, or about 150 mg, or a range between any two preceding values. In some aspects, the amount of vibegron administered per day is about 70 mg to about 80 mg. In some aspects, the amount of vibegron administered per day is about 70 mg, about 71 mg, about 72 mg, about 73 mg, about 74 mg, about 75 mg, about 76 mg, about 77 mg, about 78 mg, about 79 mg, or about 80 mg, or a range between any two preceding values.

In some aspects, vibegron is administered once per day, twice per day, or three times per day. In some aspects, vibegron is administered once per day.

The present disclosure provides a method of treating overactive bladder, the method comprising orally administering to a patient in need thereof a therapeutically effective amount of vibegron, wherein the patient is also receiving digitalis glycoside, and wherein the method further comprises monitoring serum digitalis glycoside concentrations in the patient; and titrating the digitalis glycoside dose in the patient to obtain a desired clinical effect. In some aspects, the patient is administered vibegron and is concomitantly receiving, taking or otherwise being exposed to a digitalis glycoside. In some aspects the digitalis glycoside is digoxin.

In some aspects, the patient is administered about 75 mg of vibegron per day and is concomitantly receiving, taking or otherwise being exposed to a digitalis glycoside. In some aspects the digitalis glycoside is digoxin. In certain aspects, the patient experiences an increase in the Cₘₐₓ of digoxin. In certain aspects, the patient experiences an increase in the AUC of digoxin. In some aspects, the patient experiences an increase in both the Cₘₐₓ and the AUC of digoxin.

Digoxin can be difficult to use as it can have a very narrow window between therapeutic and toxic concentrations and can also have a complex pharmacokinetic profile. In certain patients, digoxin is readily absorbed but blood levels are high for the first few hours after administration due to the time it takes to distribute from the blood to the tissues. In some aspects, the digoxin is administered at bedtime. In certain aspects, the digoxin is administered at bedtime and the blood level is measured in the morning. In some aspects, the blood level is measured in the morning to relate serum and digoxin tissue levels. In some aspects, the serum digoxin level is measured in the morning at least 5 days after beginning or altering the dose of digoxin. In some aspects, the serum digoxin level is measured in the morning at least 6 days after beginning or altering the dose of digoxin. In some aspects, the serum digoxin level is measured in the morning at least one week after beginning or altering the dose of digoxin. In some aspects, the serum digoxin level is measured about 2 weeks after the patient has been taking a steady digoxin dose. In some aspects, the serum digoxin level is measured about 3 weeks after the patient has been taking a steady digoxin dose. In some aspects, the serum digoxin level is measured about 4 weeks after the patient has been taking a steady digoxin dose.

In some aspects, the serum level of digoxin is less than 1.2 ng/mL. In some aspects, the serum level of digoxin is from about 0.3 ng/mL to about 1.2 ng/mL. In some aspects, the serum level of digoxin is from about 0.4 ng/mL to about 1.1 ng/mL. In some aspects, the serum level of digoxin is from about 0.5 ng/mL to about 1.0 ng/mL. In some aspects, the serum level of digoxin is from about 0.5 ng/mL to about 0.9 ng/mL.

In certain aspects, the dosage of digoxin is titrated based on the digoxin serum level. In some aspects, the dosage of digoxin is reduced based on the digoxin serum level. In some aspects, the dosage of digoxin is increased based on the digoxin serum level. In some aspects the dosage of digoxin is maintained based on the digoxin serum level.

In some aspects, the vibegron is discontinued and the digoxin serum level is monitored for an additional period of time beyond the discontinuation. In some aspects, the digoxin serum level is monitored for an additional week from discontinuation. In some aspects, the digoxin serum level is monitored for an additional two weeks from discontinuation. In some aspects, the digoxin serum level is monitored for an additional three weeks from discontinuation.

In some aspects, the patient has the symptoms of urgency urinary incontinence, urinary urgency, and urinary frequency.

In some aspects, the patient has one or more symptoms of urgency urinary incontinence (or urge urinary incontinence), urinary urgency, urinary frequency and nocturia.

In some aspects, the patient is a mammal. In some aspects the patient is a human or an animal. In some aspects, the patient is a human.

In some aspects, the method comprises crushing a pharmaceutical unit dose composition comprising vibegron before administration to a patient. In some aspects, the patient is orally administered a crushed pharmaceutical unit dose comprising vibegron.

In some aspects, the patient has received prior OAB therapy. In some aspects, the patient has not received prior OAB therapy.

The present disclosure also provides:

(1) Vibegron for use in treating overactive bladder, wherein a therapeutically effective amount of vibegron is to be orally administered to a subject in need thereof, wherein the subject is concomitantly receiving digoxin, and wherein additionally:
a. the serum digoxin concentrations in the subject are to be monitored; and
b. in response to said monitoring, the digoxin dose in the subject is either to be maintained or titrated to obtain a desired clinical effect.

(2) Vibegron for use in treating overactive bladder, wherein a therapeutically effective amount of vibegron is to be orally administered to a subject in need thereof, wherein the subject is concomitantly receiving digoxin, and wherein additionally:
a. the serum digoxin concentrations in the subject are to be monitored;
b. in response to said monitoring, the digoxin dose in the subject is either to be maintained or titrated to obtain a desired clinical effect;
c. the administration of vibegron is to be discontinued;
d. the serum digoxin concentrations in the subject are to be monitored; and
e. in response to said monitoring, the digoxin dose in the subject is either to be maintained or titrated to obtain a desired clinical effect.

(3) Vibegron for use in treating overactive bladder, wherein a therapeutically effective amount of vibegron is to be administered orally to a subject in need thereof, wherein the subject is concomitantly receiving digoxin, and wherein the subject's digoxin dose is to be titrated based on the subject's serum digoxin concentration.

(4) Vibegron for use in treating overactive bladder, wherein a therapeutically effective amount of vibegron is to be administered orally to a subject in need thereof, wherein the subject is concomitantly treated with digoxin, and wherein the subject's digoxin serum level is one that results in a desired digoxin clinical effect.

(5) Vibegron for use according to any one of (1) to (4), wherein the therapeutically effective amount of vibegron is between about 50 mg/day and about 150 mg/day.

(6) Vibegron for use according to any one of (1) to (5), wherein the therapeutically effective amount of vibegron is about 50 mg/day, about 75 mg/day, about 100 mg/day, or about 150 mg/day.

(7) Vibegron for use according to any one of (1) to (6), wherein the therapeutically effective amount of vibegron is 75 mg/day.

(8) Vibegron for use according to any one of (1) to (7), wherein the subject has a symptom selected from the group of urgency urinary incontinence, urinary urgency, urinary frequency, and combinations thereof.

(9) Vibegron for use according to (8), wherein the subject has the symptoms urgency urinary incontinence, urinary urgency, and urinary frequency.

(10) Vibegron for use according to any one of (1) to (9), wherein the subject is a human.

(11) Vibegron for use according to (10), wherein the human is a female.

(12) Vibegron for use according to (10), wherein the human is a male.

(13) Vibegron for use according to any one of (10) to (12), wherein the human is over the age of 65 years.

(14) Vibegron for use according to any one of (1) to (13), wherein vibegron is to be administered once per day.

(15) Vibegron for use according to (14), wherein vibegron is to be administered with a meal.

(16) Vibegron for use according to (14), wherein vibegron is to be administered without a meal.

(17) Vibegron for use according to any one of (1) to (16), wherein vibegron is to be administered as a free base.

(18) Vibegron for use according to any one of (1) to (16), wherein vibegron is to be administered as a pharmaceutically acceptable salt thereof.

(19) Vibegron for use according to any one of (1) to (18), wherein vibegron has onset of action of about 4 weeks, about 3 weeks, or about 2 weeks.

(20) Vibegron for use in treating overactive bladder, wherein 75 mg of vibegron per day is to be administered orally to a subject in need thereof, wherein the subject is concomitantly receiving digoxin, and wherein additionally:
a. the serum digoxin concentrations in the subject are to be monitored; and
b. in response to said monitoring, the digoxin dose in the subject is either to be maintained or titrated to obtain a desired clinical effect.

(21) Vibegron for use in treating overactive bladder, wherein 75 mg of vibegron per day is to be administered orally to a subject in need thereof, wherein the subject is concomitantly receiving digoxin, and wherein additionally:
a. the serum digoxin concentrations in the subject are to be monitored;
b. in response to said monitoring, the digoxin dose in the subject is either to be maintained or titrated to obtain a desired clinical effect;
c. the administration of vibegron is to be discontinued;
d. the serum digoxin concentrations in the subject are to be monitored; and
e. in response to said monitoring, the digoxin dose in the subject is either to be maintained or titrated to obtain a desired clinical effect.

(22) Vibegron for use in treating overactive bladder, wherein 75 mg of vibegron per day are to be administered orally to a subject in need thereof, wherein the subject is concomitantly receiving digoxin, and wherein the subject's digoxin dose is to be titrated based on the subject's serum digoxin concentration.

(23) Vibegron for use in treating overactive bladder, wherein 75 mg of vibegron per day are to be administered orally to a subject in need thereof, wherein the subject is concomitantly treated with digoxin, and wherein the subject's digoxin serum level is one that results in a desired digoxin clinical effect.

(24) Vibegron for use in treating overactive bladder, wherein 75 mg of vibegron per day is to be administered orally to a subject in need thereof, wherein the subject is concomitantly receiving digoxin, and wherein additionally:
a. the serum digoxin concentrations in the subject are to be monitored before administering vibegron;
b. the serum digoxin concentrations in the subject are to be monitored during concomitant administration of vibegron; and
c. in response to said monitoring, the digoxin dose in the subject is either to be maintained or titrated to obtain a desired clinical effect.

(25) Vibegron for use in treating overactive bladder, wherein 75 mg of vibegron per day is to be administered orally to a subject in need thereof, wherein the subject is concomitantly receiving digoxin, and wherein additionally:
a. the serum digoxin concentrations in the subject are to be monitored before administering vibegron;
b. the serum digoxin concentrations in the subject are to be monitored during concomitant administration of vibegron; and
c. in response to said monitoring, the digoxin dose in the subject is either to be maintained or titrated to obtain a desired clinical effect;
d. the administration of vibegron is to be discontinued;
e. the serum digoxin concentrations in the subject are to be monitored; and
f. in response to said monitoring, the digoxin dose in the subject is either to be maintained or titrated to obtain a desired clinical effect.

### EXAMPLES

### Example 1: Study to Assess the Effects of Vibegron on the Single-Dose Pharmacokinetics of Digoxin in Healthy Subjects

An open-label, single-dose and multiple-dose, two-period, two-treatment single-sequence drug-drug interaction study was conducted. All subjects received Treatment A during Period 1 and Treatment B during Period 2.

Treatment A (Period 1):

Day 1: Oral, 0.25 mg single dose of Toloxin® (digoxin) after an overnight fast of at least 8 hours.

Treatment B (Period 2):

Day 1: Oral, 150 mg single dose of vibegron administered after an overnight fast of at least 8 hours.

Day 2: Oral, 0.25 mg single dose of Toloxin® (digoxin) administered with 100 mg of vibegron after an overnight fast of at least 8 hours.

Days 3 to 6: Oral, 100 mg single dose of vibegron administered once daily after an overnight fast of at least 8 hours.

### Example 2: LC-MS/MS Methods for Analysis of Levels of Digoxin Assessed in Drug-Drug Interaction Studies

This method is applicable to the quantitation of digoxin within a nominal range of 0.0100 to 10.0 ng/mL and requires a 150-μL human plasma aliquot containing dipotassium EDTA.

Samples were stored in polypropylene tubes and kept frozen at approximately −20° C. prior to analysis. A 150-μL matrix aliquot was fortified with 20 μL of 40.0 ng/mL internal standard working solution. Analytes were isolated using an Isolute SLE+ (200-mg) supported liquid extraction 96-well plate and eluted with 800 μL of methylene chloride. The eluate was evaporated under a nitrogen stream at approximately 45° C., and the remaining residue was reconstituted with 250 μL of 90:10:40:0.7 water/methanol/acetonitrile/1.0 M ammonium acetate, v/v/v/v. The final extract was analyzed via HPLC with column-switching and MS/MS detection using positive ion electrospray. Extracts were analyzed by HPLC MS/MS using positive ion electrospray. Additional parameters are shown in Tables 1 and 2.

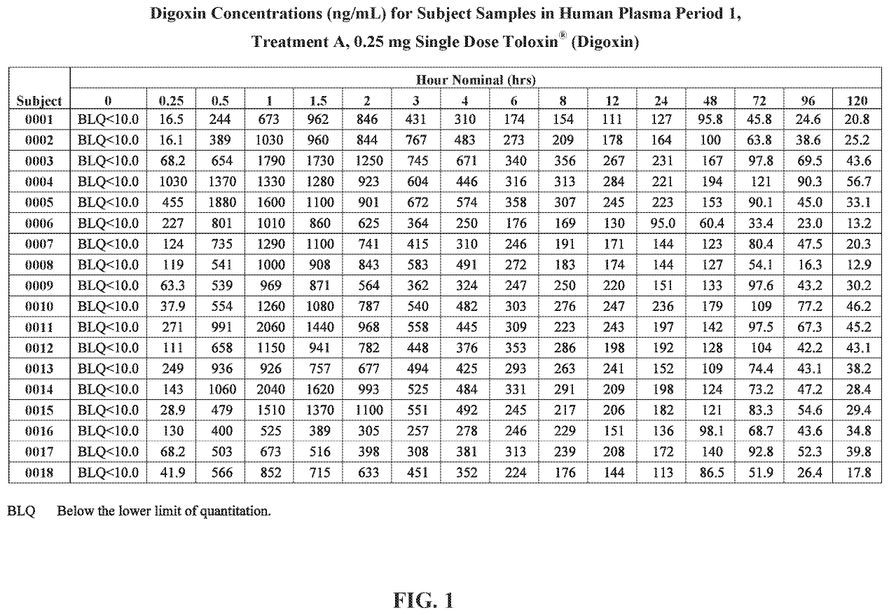

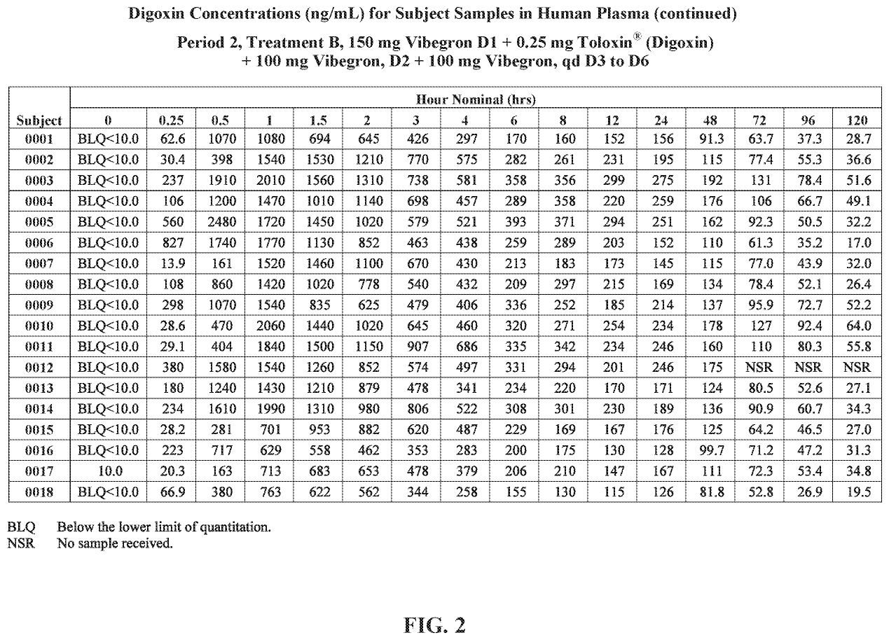

Results of the subject serum digoxin concentration versus collection time points for each treatment were generated using PPD's Assist LIMS database. The serum digoxin concentration data are presented in FIG. 1 and FIG. 2.

TABLE 1
Summary of Method Validation of LC-MS/MS to Measure
Digoxin in Plasm

| Validated assay | 10 to 10000 pg/mL | |
|---|---|---|
| range | | |
| Materials used for | Mobile Phase: 90:10:40:0.7 water/methanol/ | |
| standard | acetonitrile/1.0M ammonium acetate, | |
| calibration curve | v/v/v/v. | |
| and | 800 μL of methylene chloride | |
| concentration | Concentrations: 0.01, 0.20, 0.40, 0.15, | |
| | 0.60, 2.50, 8.00, 10.00 ng/ml | |
| Materials used for | Same materials as above. | |
| quality | Concentrations: 10.0, 25.0, 75.0, 300, | |
| controls (QCs) and | 1250, 7500 pg/mL | |
| concentration | | |
| Source and lot of | Digoxin lot number: O0B096 | |
| reagents | Internal standard [D3]Digoxin lot | |
| | number: S-1231-049A4 | |
| Regression model | Linear; 1/x² | |
| & weighting | | |
| Validation | Method validation summary | |
| parameters | | |
| Standard | Number of standard calibrators | 8 |
| calibration curve | from LLOQ to ULOQ | |
| performance | Cumulative accuracy (% bias) | −3.06 to 1.57 |
| during accuracy & | from LLOQ to ULOQ | |
| precision | Cumulative precision (% CV) | ≤7.57 |
| | from LLOQ to ULOQ | |
| QCs performance | Accuracy (% bias) in QCs | |
| during | Within run: | −17.7 to 21.2 |
| accuracy & | Between run: | −3.99 to 2.71ª |
| precision | | |
| | Precision (% CV) in QCs | |
| | Within run: | ≤ 29.5^{b} |
| | Between run: | ≤ 19.9ª |

| | |
|---|---|
| Selectivity & matrix | Number of total lots tested: 14 matrix |
| effect | Range of observed bias: not |
| | reported the sensitivity or |
| | State any issue: There were no significant |
| | suppression effects indicated that |
| | could compromise accuracy of |
| | the assay. |
| Interference & | Number of total lots tested: 12 |
| specificity | Range of observed bias: not reported |
| | State any issue: none |
| Dilution linearity | Highest concentration tested: Not applicable |
| | Number of dilution factors: 10 |
| Bench-top/process | 24 hours at room temperature |
| stability | |
| Freeze-Thaw | 5 freeze-thaw cycles from −20° C. or −70° C. |
| stability | |
| Long-term storage | 286 days at −20° C., 286 days at −70° C. |
| Carry over | There were contributions from |
| | chromatographic peaks, at |
| | the expected retention time |
| | of the analyte in the blank |
| | samples, greater than 20% of |
| | the mean analyte response |
| | for the LLOQ calibration |
| | standards and quality controls |
| | ranging from 20.7 to 38.4%. |
| | The observed carryover does |
| | not impact the integrity of the data. |
| Recovery | 82.0, 90.6, and 89.7% |

^{a}Digoxin:
Note
that accuracy and precision values for one run for digoxin were rejected from evaluation of between-assay accuracy and precision due to unacceptable quality controls. The unacceptability of the run was thought due to an unidentified problem with preparation of calibration curve for run.
^{b}Digoxin: According to pre-defined specifications for assay validation, the intra-assay % CV for the replicate LLQ QC determinations should not exceed 20%, and the mean accuracy should be within 20% of the theoretical concentrations. Of the 9 runs conducted for evaluation of within-assay accuracy and precision for digoxin, the percent difference from theoretical was >20% for the LLQ QC sample for one run and the percentage % CV was >20% for the LLQ QC sample for another run. As the % CV and % bias for LLQ QC runs were acceptable for 7 of the 9 runs, and the values for the low concentration QC samples were also acceptable, the data from runs were accepted.

TABLE 2
Performance of LC-MS/MS Validated Analytical Methods
in Clinical Study: Digoxin in Plasma

| | |
|---|---|
| Assay passing | 5 of 5 (100%) plasma sample runs passed |
| rate | |
| Standard curve | Cumulative bias range: −1.73 to 1.48% |
| performance | Cumulative precision: ≤7.26% CV |
| QC performance | Cumulative bias range: −9.02 to 5.36% |
| | Cumulative precision: ≤10.7% CV |
| Method | Not determined |
| reproducibility | |
| Study sample | Maximum sample storage duration |
| analysis/stability | from collection to analysis: 55 days at |
| | −20° C. Coverage: 286 days at −20° C. |

### Example 3: Clinical Results of Study to Assess the Effects of Vibegron on the Single-Dose Pharmacokinetics of Digoxin in Healthy Subjects

Tables 3 and 4 show the pharmacokinetic results of the study. As shown in each table, the AUC_{0-inf} and Cₘₐₓ Geometric Mean Ratio and Arithmetic Mean Ratio of digoxin when co-administered with vibegron was 111% and 121%, respectively. As these numbers are contained within the 80-125% bioequivalence range, they suggest that vibegron does not influence digoxin pharmacokinetics to a clinically significant degree. However, given digoxin's narrow therapeutic window and the observed increase of the half-life (T_{1/2}) of digoxin in the group receiving digoxin+vibegron as compared to the group receiving digoxin alone (see Table 4), the digoxin serum level should be monitored, and the dose of digoxin titrated, if needed, in order to obtain the desired clinical effect.

TABLE 3
Summary of Pharmacokinetic Parameters of Co-Administered
Drug (Conmed) in the Presence of Vibegron

| | | | | | | | Ratio (with/without | |
|---|---|---|---|---|---|---|---|---|
| | | | | | | | Vibegron) of Conmed | |
| | | | | | Geometric Mean | | Pharmacokinetic | |
| | Dose of | Dose of | | | (95% CI) of Conmed | | Parameters; No | |
| | Conmed | Vibegron | | | Conmed | Conmed + | Effect = 1.00 | |
| Conmed | (mg) | (mg) | n | | alone | Vibegron | GMR | (90% CI) |
| Digoxin | 0.25 mg | 100 mg QD | 18 | AUC | 16600 | 18400 ^{a} | 1.11 | (1.03, 1.19) |
| | single dose | | | | (14600, 19200) | (16200, 21000) | | |
| | | | | Cₘₐₓ | 1160 | 1410 | 1.21 | (1.09, 1.35) |
| | | | | | (965, 1400) | (1170, 1700) | | |

GMR = Geometric Means Ratio; CI = confidence interval; Concentration data converted from molar to ng/mL (molecular weight of vibegron = 444.5)
^{a} N = 17

TABLE 4
Summary of Pharmacokinetic Parameters of Co-Administered
Drug in the Presence of Vibegron (Arithmetic Mean)

| | Co-Administered | | | | | | |
|---|---|---|---|---|---|---|---|
| Population/ | Drug/Route of | | | Mean (SD) Parameter^{a} of Co-Administered Drug | | | |
| No. of | Administration/ | Dose/No. of | | Cₘₐₓ | | AUC(O-∞) | |
| Subjects | Dosage Form | Subjects | Regimen | (pg/mL) | Tₘₐₓ | pg · h/mL) | T_{1/2} (h) |
| Healthy | Digoxin/oral/tablet | 0.25 mg single | Digoxin | 1240 | 1.00 | 17200 | 36.8 |
| subjects/18 | | dose/18 | | (454) | (0.5-1.50) | (4420) | (7.1) |
| | | 0.25 mg × 1 dose | Digoxin with | 1490 | 1.00 | 18900^{g} | 39.4^{g} |
| | | and 150 mg × 1 | vibegron | (499) | (0.5-1.50) | (4840) | (6.9) |
| | | dose + 100 mg × | 100 mg × | | | | |
| | | 1 dose/18 | 5 doses | | | | |

^{a}Tₘₐₓ presented as median (range)
^{g}N = 17

Having now fully described this invention, it will be understood by those of ordinary skill in the art that the same can be performed within a wide and equivalent range of conditions, formulations and other parameters without affecting the scope of the invention or any aspect thereof.

Other aspects of the invention will be apparent to those skilled in the art from consideration of the specification and practice of the invention disclosed herein. It is intended that the specification and examples be considered as exemplary only, with a true scope and spirit of the invention being indicated by the following claims.

All patents, patent applications, and other publications cited herein are fully incorporated by reference herein in their entirety.

What is claimed is:

1. A method of treating overactive bladder, the method comprising orally administering to a patient in need thereof a therapeutically effective amount of vibegron, wherein the patient is concomitantly receiving digoxin, and wherein the method further comprises:
a. monitoring serum digoxin concentrations in the patient; and
b. in response to said monitoring, either maintaining or titrating the digoxin dose in the patient to obtain a desired clinical effect.

2. A method of treating overactive bladder, the method comprising orally administering to a patient in need thereof a therapeutically effective amount of vibegron, wherein the patient is concomitantly receiving digoxin, and wherein the method further comprises:
a. monitoring serum digoxin concentrations in the patient;
b. in response to said monitoring of step (a), either maintaining or titrating the digoxin dose in the patient to obtain a desired clinical effect;
c. discontinuing administration of vibegron;
d. monitoring serum digoxin concentrations in the patient after said discontinuation; and
e. in response to said monitoring of step (d), either maintaining or titrating the digoxin dose in the patient to obtain a desired clinical effect.

3. A method of treating overactive bladder, the method comprising orally administering to a patient in need thereof a therapeutically effective amount of vibegron, wherein the patient is concomitantly receiving digoxin, and wherein the patient's digoxin dose is titrated based on the patient's serum digoxin concentration.

4. A method of treating overactive bladder, the method comprising orally administering to a patient in need thereof a therapeutically effective amount of vibegron, wherein the patient is concomitantly treated with digoxin, and wherein the patient's digoxin serum level is one that results in a desired digoxin clinical effect.

5. The method of any one of claims 1 to 4, wherein the therapeutically effective amount of vibegron is between about 50 mg/day and about 150 mg/day.

6. The method of any one of claims 1 to 5, wherein the therapeutically effective amount of vibegron is about 50 mg/day, about 75 mg/day, about 100 mg/day, or about 150 mg/day.

7. The method of any one of claims 1 to 6, wherein the therapeutically effective amount of vibegron is 75 mg/day.

8. The method of any one of claims 1 to 7, wherein the patient has a symptom selected from the group of urgency urinary incontinence, urinary urgency, urinary frequency, and combinations thereof.

9. The method of claim 8, wherein the patient has the symptoms of urgency urinary incontinence, urinary urgency, and urinary frequency.

10. The method of any one of claims 1 to 9, wherein the patient is a human.

11. The method of claim 10, wherein the human is a female.

12. The method of claim 10, wherein the human is a male.

13. The method of any one of claims 10 to 12, wherein the human is over the age of 65 years.

14. The method of any one of claims 1 to 13, wherein vibegron is administered once per day.

15. The method of claim 14, wherein vibegron is administered with a meal.

16. The method of claim 14, wherein vibegron is administered without a meal.

17. The method of any one of claims 1 to 16, wherein vibegron is administered as a free base.

18. The method of any one of claims 1 to 16, wherein vibegron is administered as a pharmaceutically acceptable salt thereof.

19. The method of any one of claims 1 to 18, wherein vibegron has onset of action of about 4 weeks, about 3 weeks, or about 2 weeks.

20. A method of treating overactive bladder, the method comprising orally administering to a patient in need thereof 75 mg of vibegron per day, wherein the patient is concomitantly receiving digoxin, and wherein the method further comprises:
a. monitoring serum digoxin concentrations in the patient; and
b. in response to said monitoring, either maintaining or titrating the digoxin dose in the patient to obtain a desired clinical effect.

21. A method of treating overactive bladder, the method comprising orally administering to a patient in need thereof 75 mg of vibegron per day, wherein the patient is concomitantly receiving digoxin, and wherein the method further comprises:
a. monitoring serum digoxin concentrations in the patient;
b. in response to said monitoring of step (a), either maintaining or titrating the digoxin dose in the patient to obtain a desired clinical effect;
c. discontinuing administration of vibegron;
d. monitoring serum digoxin concentrations in the patient after said discontinuation; and
e. in response to said monitoring of step (d), either maintaining or titrating the digoxin dose in the patient to obtain a desired clinical effect.

22. A method of treating overactive bladder, the method comprising orally administering to a patient in need thereof 75 mg of vibegron per day, wherein the patient is concomitantly receiving digoxin, and wherein the patient's digoxin dose is titrated based on the patient's serum digoxin concentration.

23. A method of treating overactive bladder, the method comprising orally administering to a patient in need thereof 75 mg of vibegron per day, wherein the patient is concomitantly treated with digoxin, and wherein the patient's digoxin serum level is one that results in a desired digoxin clinical effect.

24. A method of treating overactive bladder in a patient, the method comprising:
a. measuring serum digoxin concentrations in the patient;
b. orally administering to the patient 75 mg of vibegron per day, wherein the patient is concomitantly receiving digoxin;
c. monitoring serum digoxin concentrations in the patient; and
d. in response to said monitoring of step (c), either maintaining or titrating the digoxin dose in the patient to obtain a desired clinical effect.

25. A method of treating overactive bladder in a patient, the method comprising:
a. measuring serum digoxin concentrations in the patient;
b. orally administering to the patient 75 mg of vibegron per day, wherein the patient is concomitantly receiving digoxin;
c. monitoring serum digoxin concentrations in the patient;
d. in response to said monitoring of step (c), either maintaining or titrating the digoxin dose in the patient to obtain a desired clinical effect;
e. discontinuing administration of vibegron;
f. monitoring serum digoxin concentrations in the patient after said discontinuation; and
g. in response to said monitoring of step (f), either maintaining or titrating the digoxin dose in the patient to obtain a desired clinical effect.
